(19) United States
(12) Patent Application Publication
(10) Pub. No.: US 2002/0052584 A1
(43) Pub. Date: May 2, 2002

(54) Absorbent article provided with a belt

(76) Inventor: Monica Forgar, Blackevagen (SE)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 10/026,710

(22) Filed: Dec. 27, 2001

Related U.S. Application Data
(63) Continuation of application No. PCT/SE00/01287, filed on Jun. 19, 2000.

(30) Foreign Application Priority Data
Jun. 29, 1999 (SE) 9902448-1

(51) Int. Cl. A61F 13/15; A61F 13/20

(52) U.S. Cl. 604/358

(57) ABSTRACT

Absorbent article such as a diaper and an incontinence guard comprising a liquid permeable topsheet (2), a liquid impermeable backsheet (3) and an absorbent body (4) enclosed therebetween, said article having a front portion (5), a rear portion (6) and a crotch portion (7) therebetween, and further is provided with a belt (9) attached to or intended to be attached to the rear portion (6) of the article and to the front portion (5) of the article, in such a way that the article will assume a pantlike shape, where the belt (9) forms a part of the waist portion of the pant. The belt (9) comprises a flexible laminate comprising an elastic carrier material (11), which at least on a part of the side intended to form the outside of the belt is covered with a nonwoven material (12) having a basis weight of at least 15 and preferably at least 20 g/m² and which may serve as attachment surface for attachment means (8,10) both in the form of adhesive tape and hook-and-loop type fasteners, at which the attachment surface also admits refastening of said attachment means.

### TECHNICAL FIELD

The present invention refers to an absorbent article such as a diaper and an incontinence guard comprising a liquid permeable topsheet, a liquid impermeable backsheet and an absorbent body enclosed therebetween, said article having a front portion, a rear portion and a crotch portion therebetween, and further is provided with a belt attached to or intended to be attached to the rear portion of the article and to the front portion of the article, in such a way that the article will assume a pantlike shape, where the belt forms a part of the waist portion of the pant.

### BACKGROUND OF THE INVENTION

Diapers and incontinence guards for incontinent adults usually have a garment portion holding an absorbent body in place against the user's body and attachment means which hold the garment portion in place also when the user is moving. A common type of attachment means are adhesive tapes or hook-and-loop fasteners which directly attach the front and rear portions of the absorbent article to each other. It is further known, through e g EP-A-0 287 388, EP-A-0 409 307, EP-A-0 605 012 and FR-A-2 586 558, to attach the front and rear portions of the article by means of a belt, at which the possibilities to adjust the fit are improved. Depending on type of attachment means different types of attachment surfaces are used which are intended to cooperate with the attachment means.

A problem with these belts is that they easily cause skin irritations to the user, due to that the belt is in direct contact with the skin of the wearer and has to be tightened relatively strongly in order to have a satisfactory fit and security against leakage of the diaper or incontinence guard. By the tight contact and friction between the belt and the skin there will be a mechanic wear of the skin which gives rise to irritation and even skin injuries.

### THE OBJECT AND MOST IMPORTANT FEATURES OF THE INVENTION

The object of the invention is to provide a belt for absorbent articles which is kind to the skin and by that does not give rise to skin irritations and injuries and in which the belt material per se can cooperate with different kinds of attachment means for providing a flexible fit of the absorbent article. This has been solved by the fact that the belt comprises a flexible laminate comprising an elastic carrier material, which at least on a part of the side intended to form the outside of the belt is covered with a nonwoven material having a basis weight of at least 15 and preferably at least 20 g/m² and which may serve as attachment surface for attachment means both in the form of adhesive tape and hook-and-loop type fasteners, at which the attachment surface also admits refastening of said attachment means.

The nonwoven material which is intended to form the outside of the belt mainly comprises continuous filaments, such as spunbond material or meltblown material.

The elastic carrier material can be an elastic film, which preferably is perforated, or an elastic nonwoven material.

The elastic carrier material is preferably on the side intended to form the inside of the belt covered with a soft skin friendly nonwoven material.

According to a preferred embodiment at least the nonwoven material that is intended to form the outside of the belt is joined with the elastic material in a pattern so that a three-dimensional surface structure is obtained at least on one side of the laminate. By that an improved attachment surface is obtained for a hook-and-loop type fastener.

### DESCRIPTION OF DRAWINGS

The invention will in the following be closer described with reference to an embodiment shown in the accompanying drawings.

FIG. 1 shows schematically a plan view of an absorbent article according to the invention.

FIG. 2 shows schematically an exploded view of a laminate according to the invention.

FIG. 3 shows schematically the laminate seen from one side thereof.

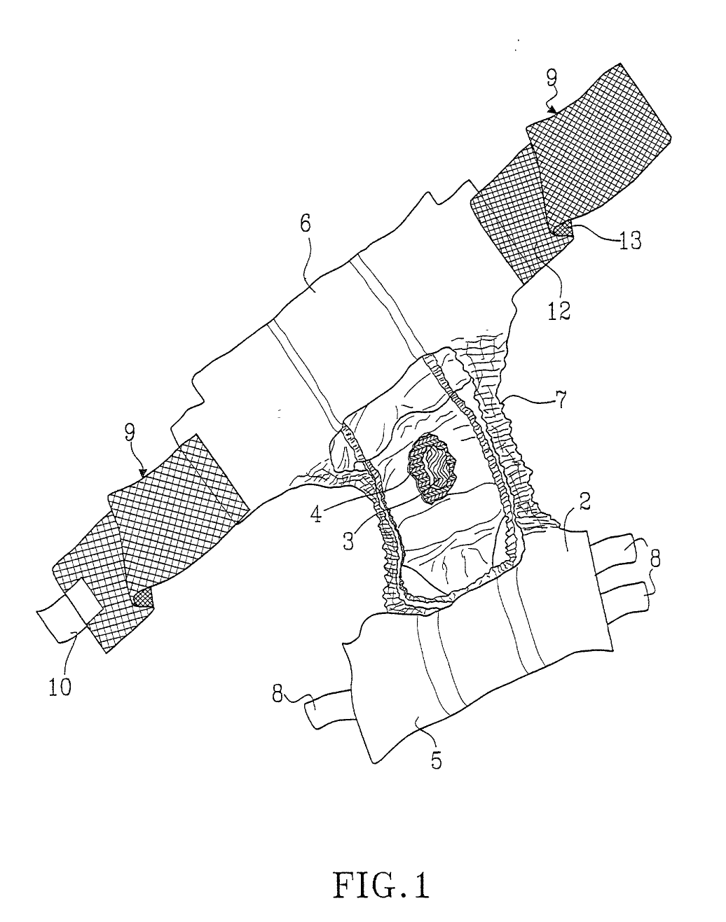

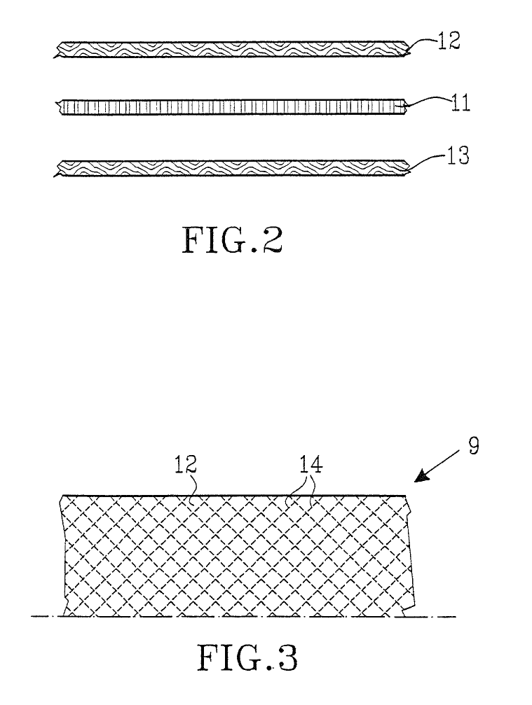

### DESCRIPTION OF AN EMBODIMENT

The drawing shows an embodiment of a diaper or incontinence guard 1 comprising a liquid permeable topsheet 2, a liquid impermeable backsheet 3 and an absorbent body 4 enclosed therebetween. The liquid permeable topsheet 2 can consist of a nonwoven material, e g a spunbond material of continuous filaments, a meltblown material or a bonded carded fibrous web. The liquid impermeable backsheet 3 may consist of a plastic film, a nonwoven material coated with a liquid impervious material or a hydrophobic nonwoven material which resists liquid penetration.

The topsheet 2 and the backsheet material 3 has a somewhat greater extension in the plane than the absorbent body 4 and extends outside the edges thereof. The layers 2 and 3 are connected to each other within the projecting portions thereof, e g by gluing or welding by heat or ultrasonic.

The absorbent body 4 can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbents in an absorbent body. It is also common to have absorbent bodies comprising layers of different material with different properties with respect to liquid acquisition capacity, liquid distribution capacity and storage capacity. It is well-known to the person skilled in the art and does therefore not have to be described in detail. The thin absorbent bodies which are common in for example baby diapers and incontinence guards often comprise a compressed mixed or layered structure of cellulosic fluff pulp and superabsorbent.

The diaper is intended to enclose the lower part of the wearer's trunk like a pair of absorbent pants. It comprises a front portion 5 intended during use to be worn on the front part of the user's body, a rear portion 6 intended during use to be worn on the rear part of the user's body, and a more narrow crotch portion 7 located between the front and rear portions and which is intended to be worn in the crotch part of the user between the legs. The front portion 5 is provided with a pair of adhesive tape tabs 8 or other type of attachment means such as hooks-and-loop type fasteners.

A pair of belt portions 9 are with one end attached, e g glued or ultrasonically welded to the rear part 5 of the diaper. The belt portions 9 are with their opposite ends intended to be fastened together, e g by a tape tab 10 which is taped to the outside of the opposite belt portion. Instead of tape there may be another type of optional attachment means, such as a hook-and-loop type fastener. The tape tabs 8 or corresponding attachment means of the front portion 5 are intended to be attached against the outside of the belt portions 9 in order to fasten together the diaper to the desired pantlike shape.

The width of the belt portions should be between 5 and 20 cm, preferably between 7 and 15 cm.

The belt portions 9 consist of an elastic laminate of an inner elastic material 11, which on both sides are covered by nonwoven materials 12, 13, one of which 12 is intended to form the outside of the belt and the other 13 is intended to form the inside of the belt, i e the side which is intended to contact the skin of the wearer. These nonwoven materials may be the same or different. The elastic carrier material 11 may, if it is soft and skin friendly enough, form the inside of the belt, at which the additional inside material 13 is eliminated.

At least the nonwoven material 12 that is intended to form the outside of the belt should be of a kind that can serve as an attachment surface for attachment means in the form of adhesive tapes as well as hook-and-loop type fasteners. These attachment means are on one hand the attachment member 10 at one belt portion 9, which is intended to be fastened to the outside of the opposite belt portion and on the other hand the attachment means 8 at the front portion of the diaper, said attachment means 8 are intended to be fastened to the outside of the belt for fastening together the diaper to the desired pant shape.

The most commonly occurring attachment means are either tape tabs or hook-and-loop type fasteners. As an attachment surface for a tape there is normally used a smooth or embossed plastic film, while a nonwoven material generally functions well as an attachment surface for the hooks in a hook-and-loop type fastener. It has now according to the invention proved that certain types of nonwoven materials also functions well as an attachment surface for a tape. It should also be possible to open and refasten the tape.

The nonwoven material should mainly consist of continuous filaments, such as spunbond material or meltblown material of for example polypropylene, polyethylene or bicomponent fibers. The basis weight of the nonwoven material which should form the attachment surface for a tape should be at least 15 and preferably at least 20 g/m², and be laminated to a carrier material, in this case the elastic material 11. Possibly the nonwoven material 12 does not need to cover the outside of the entire belt, but only the portions thereof which are intended to be used as a refastenable attachment surface for the attachment means 8 and 10.

Due to the fact that the outside of the laminate functions as an attachment surface for tapes as well as for the hooks of a hook-and-loop type fastener it is possible to use different types of attachment means 8 and 10, i e the attachment means 8 may be for example a tape while the attachment means 10 may be the hooks of a hook-and-loop type fastener. A great freedom in choice is given both regards the fit of the belt, since preferably the entire outside thereof can function as a refastenable attachment surface for the attachment means 8 and 10.

The nonwoven material that is intended to form the inside of the belt, i e directly contact the skin of the wearer, should be soft and skin friendly. A suitable nonwoven material may be the same as used as outside material 12, i e a spunbond material or a meltblown material of for example polypropylene or polyethylene. Bicomponent fibers may also be used. Another suitable nonwoven material is a carded thermobonded material of for example polypropylene, polyester- or bicomponent fibers.

As an elastic carrier material 11 there can be used an elastic film, e g of styrene-butadiene-styrene. The film can optionally be provided with holes, e g be perforated, so that it is breathable, in order to make the belt more comfortable and skin friendly. The thickness of the film is preferably in the interval 30-200 μm. Alternatively an elastic nonwoven material can be used as an elastic carrier material.

The elastic material 11 and the nonwoven material 12,13 are joined together in any suitable way, e g by gluing, heat calendering, ultrasonic welding or in another way. According to a preferred embodiment at least the nonwoven material 12 which is intended to form the outside of the belt is joined to the elastic material 11 in a pattern 14 so that a three-dimensional surface structure is formed on said side of the laminate, due to that the material has been compressed just opposite the bonding sites. Such a pattern structure 14 can for example be obtained by ultrasonic welding or heat calendering. Alternatively this may also be obtained by gluing the material layers together in a glue pattern. An improved gripping surface for the hooks of a hook-and-loop type fastener is provided by the three-dimensional structure.

The elasticity of the belt laminate is preferably obtained by the fact that also the nonwoven material 12, 13 has a certain degree of elasticity at least in one direction, the longitudinal direction of the belt. The nonwoven materials 12, 13 can also be joined to the elastic material 11 when the latter is kept in a stretched condition, at which the laminate will be elastic also in case relatively non-elastic nonwoven materials are used.

The invention is of course not limited to the above described embodiments but can be modified within the scope of the claims.

1. Absorbent article such as a diaper and an incontinence guard comprising a liquid permeable topsheet (2), a liquid impermeable backsheet (3) and an absorbent body (4) enclosed therebetween, said article having a front portion (5), a rear portion (6) and a crotch portion (7) therebetween, and further is provided with a pair of belt portions (9) attached to the rear portion (6) of the article and which are intended to be fastened together around the waist of the wearer by belt attachment means (10) and where said front portion (5) is provided with front portion attachment means (8) intended to be attached to the belt portions (9), in such a way that the article will assume a pantlike shape, where the belt portions (9) form a part of the waist portions of the pant,
characterized in
that the belt portions (9) comprise a flexible laminate comprising an elastic carrier material (11), which at least on a part of the side intended to form the outside of the belt portions is covered with a nonwoven material (12) having a basis weight of at least 15 and preferably at least 20 μm² and which is adapted to serve as attachment surface for belt portion and front portion attachment means (8,10) both in the form of adhesive tape and hook-and-loop type fasteners, at which the attachment surface also admits refastening of said attachment means.

2. Absorbent article according to claim 1,
characterized in
that said nonwoven material (12) which is intended to form the outside of the belt mainly comprises continuous filaments, such as spunbond material or meltblown material.

3. Absorbent article according to claim 1 or 2,
characterized in
that the elastic carrier material (11) is an elastic film.

4. Absorbent article according to claim 3,
characterized in
that the film (11) is perforated.

5. Absorbent article according to claims 1 or 2,
characterized in
that the elastic carrier material (11) is an elastic nonwoven material.

6. Absorbent article according to any of the preceding claims,
characterized in
that the elastic canner material (11) on the side which is intended to form the inside of the belt is covered with a soft skin friendly nonwoven material.

7. Absorbent article according to any of the preceding claims,
characterized in
that at least the nonwoven material (12) which is intended to form the outside of the belt is joined to the elastic material (11) in a pattern (14) so that a three-dimensional surface structure is formed on said side of the laminate.
